SEQUENCE LISTING (14 sequences)

SEQ ID NO: 1 - protein, 5 aa, Homo sapiens
  MISC_FEATURE (3)..(3): Xaa = Ala or Pro
KKXXK

SEQ ID NO: 2 - protein, 234 aa, Homo sapiens
MSETAPAAPAAPAPAEKTPVKKKARKSAGAAKRKASGPPVSELITKAVAASKERSGVSLAALKKALAAAGYDVEKNNSRIKLGLKSLVSKGTLVQTKGTGASGSFKLNKKAASGEAKPKAKKAGAAKAKKPAGAAKKPKKATGAATPKKSAKKTPKKAKKPAAAAGAKKAKSPKKAKAAKPKKAPKSPAKAKAVKPKAAKPKTAKPKAAKPKKAAAKKKKLEQKLISEEDLKLN

SEQ ID NO: 3 - protein, 20 aa, Homo sapiens
TPKKAKKPAAAAGAKKAKSP

SEQ ID NO: 4 - protein, 22 aa, Homo sapiens
GAAKAKKPAGAAKKPKKATGAA

SEQ ID NO: 5 - protein, 130 aa, Homo sapiens
LVQTKGTGASGSFKLNKKAASGEAKPKAKKAGAAKAAKKPAGAAKKPKKATGAATPKKSAKKTPKKAKKPAAAAGAKKAKSPKKAKAAKPKKAPKSPAKAKAVKPKAAKPKTAKPKAAKPKKAAAKKKKL

SEQ ID NO: 6 - protein, 77 aa, Homo sapiens
LVQTKGTGASGSFKLNKKAASGEAKPKAKKAGAAKAKKPAGAAKKPKKATGAATPKKSAKKTPKKAKKPAAAAGAEF

SEQ ID NO: 7 - protein, 66 aa, Homo sapiens
KKAKKPAAAAGAKKAKSPKKAKAAKPKKAPKSPAKAKAVKPKAAKPKTAKPKAAKPKKAAAKKKEF

SEQ ID NO: 8 - protein, 55 aa, Homo sapiens
KKAKKPAAAAGAKKAKSPKKAKAAKPKKAPKSPAKAKAVKPKAAKPKTAKPKAAE

SEQ ID NO: 9 - protein, 94 aa, Homo sapiens
MSETAPAAPAAPAPAEKTPVKKKARKSAGAAKRKASGPPVSELITKAVAASKERSGVSLAALKKALAAAGYDVEKNNSRIKLGLKSLVSKGTEF

SEQ ID NO: 10 - protein, 20 aa, Homo sapiens
TPKKASSPAAAAGAKKAKSP

SEQ ID NO: 11 - protein, 20 aa, Homo sapiens
TPKKAKKPAAAAGASSAKSP

SEQ ID NO: 12 - protein, 4 aa, Homo sapiens
IEGR

SEQ ID NO: 13 - protein, 5 aa, Homo sapiens
DDDDK

SEQ ID NO: 14 - protein, 5 aa, Homo sapiens
LVPRG

(19) United States
(12) Patent Application Publication
(10) Pub. No.: US 2004/0110928 A1
(43) Pub. Date: Jun. 10, 2004

(54) Peptide conjugates for drug delivery

(76) Inventors: Andrea Crisanti, London (GB); Selma Esseghir, London (GB)

Correspondence Address: Saliwachik Lloyd & Saliwanchik, Suite A 1, 2421 NW 41st Street, Gainesville, FL, 32606-6669, US

(21) Appl. No.: 10/240,430

(22) PCT Filed: Apr. 12, 2001
(86) PCT No.: PCT/GB01/01697

(30) Foreign Application Priority Data
Apr. 12, 2000 (GB) 00090803
Feb. 2, 2001 (GB) 01026673

(51) Int. Cl. A61K 38/16; C07K 14/47

(52) U.S. Cl. 530/358

(57) ABSTRACT

A conjugate of a peptide capable of translocation across a cell membrane, and a therapeutic agent. The peptide comprises a defined amino acid sequence that provides the signal necessary for translocation.

### FIELD OF THE INVENTION

The present invention relates to the preparation of proteins as translocation agents, particularly, but not exclusively, in the form of histone-therapeutic agent conjugates.

### BACKGROUND OF THE INVENTION

Gene therapy provides the potential to cure selected genetic diseases. However, a major obstacle is the effective delivery of the gene or protein of interest to the target site. A variety of viral and non-viral vectors have been developed to deliver genes or gene products to various cells, tissues and organs by ex vivo or in vivo strategies. Among viral-based vectors, retroviruses, adenoviruses, adeno-associated viruses and herpes viruses have been most extensively studied. Among non-viral-based vectors, liposomes and cationic lipid-mediated systems have been used to introduce plasmic DNA directly into animals. However, one of the main challenges of gene therapy remains the design of effective delivery systems.

Histones have also been proposed for use as a vehicle for gene delivery via transfection. Histones are the proteins responsible for the nucleosomal organisation of chromosomes in eukaryotes. The core histones H2A, H2B, H3 and H4 form the core structure of the nucleosome, and the linker histone H1 seals two rounds of DNA at the nucleosomal core.

Zaitsev et al, Gene Therapy (1997)4, 586-592 discloses certain nuclear proteins, including histone, which can be prepared to act as DNA carriers via transfection. The example disclosed is histone H1 which is prepared in a serum-containing media with calcium ions required to obtain high transfection efficiencies. Chloroquine is also present to obtain efficient transfection. However, the presence of serum, calcium ions and chloroquine makes this formulation unsuitable for clinical applications.

Haberland et al., Biochimica et Biophysica Acta, 1999; 1445: 21-30, discloses that histones require Ca²⁺ to achieve high transfection efficiency. In the absence of Ca²⁺, chloroquine was required.

EP-A-0908521 discloses a transfection system for the transfer of nucleic acids into cells. Transfection is achieved using histones which bind to polynucleotides and then transfer the DNA into the cell.

Fritz et al., Human Gene Therapy, 1996; 7: 1395-1404, also uses DNA-binding histone to transfect DNA. However, this system also requires lipofectin to enhance transfection efficiency. Lipofectin is toxic and is generally unsuitable for therapeutic applications.

Schwartz et al., Gene Therapy, 1999; 6: 282-292, discloses a transfection system based on cationic lipids. The system requires the DNA to be transported to be first compacted using histone peptides. The compacted DNA/histone complex is then brought into contact with the cationic lipid and used in the transfection process.

WO-A-89/10134 discloses chimeric peptides for neuropeptide delivery through the blood-brain barrier. The chimeric peptides comprise a neuropeptide and a peptide capable of crossing the blood-brain barrier via receptor-mediated transcytosis. Histone is mentioned as a peptide that fulfills this criteria. The chimeric peptide is produced via chemical linkage, so that on crossing the blood-brain barrier, the linkage is broken to release the neuropeptide. The neuropeptides act on extracellular receptors to exert their therapeutic effects and do not enter the neural cells.

### SUMMARY OF THE INVENTION

The present invention is based on the surprising finding that histone proteins and other proteins or peptides comprising a specific amino acid motif can be prepared and used to translocate therapeutic agents across a cell membrane.

According to one aspect of the present invention, there is a conjugate of a peptide capable of translocating across a cell membrane, and a therapeutic or diagnostic agent, wherein the peptide comprises the amino acid sequence shown as SEQ ID NO. 1.

The conjugate preferably does not comprise, as the therapeutic agent, a neuropeptide that acts within the cell.

Surprisingly, it has been found that peptides and proteins that comprise the amino acid sequence SEQ ID NO. 1, for example histone H1, can act via translocation, to deliver a covalently bound therapeutic or diagnostic agent intracellularly. This is in contrast to the conventional use of histone in transfection of DNA (which does not depend on translocation), where the DNA is not covalently bound to the histone, and where there is no indication that histone could be used to deliver other therapeutic agents, e.g. proteins or chemical compounds, into a cell. It is also in contrast to the disclosure in WO-A-98/10134, where receptor-mediated transcytosis is used to deliver a neuropeptide across the blood-brain barrier, and there is no suggestion that intracellular delivery could be achieved. Furthermore, in contrast to conventional translocating agents such as tat, VP22 or antennapedia, the present invention permits human derived peptides (e.g. from human histone) to be used as the translocation factor, thereby reducing the risk of adverse immunological reactions that may result from the use of non-human peptides.

According to a second aspect of the invention, a therapeutic agent that exerts its therapeutic effect within a cell, is used in the manufacture of a composition to treat or diagnose a disease, wherein the agent is conjugated to a peptide that comprises the amino acid sequence identified herein as SEQ ID NO. 1.

According to a third aspect of the invention, a conjugate as defined above is used in the manufacture of a composition to treat or diagnose a disease, wherein the disease is not associated with a neurological disorder.

According to a fourth aspect of the invention, an expression vector is prepared that expresses a conjugate of the invention in the form of a fusion protein.

### DESCRIPTION OF THE INVENTION

The present invention provides conjugates with ability to transport therapeutic agents across a cell membrane to effect entry of the agent into the cell or across an intracellular compartment.

In the context of the present invention, the term “translocation” refers to the ability of an agent to cross a cellular membrane, i.e. to enter a cell. The term “transfection” refers to the delivery of a polynucleotide, e.g. DNA, to inside a cell and is usually carried out via an uptake mechanism, e.g. cell surface receptors.

The term “conjugate” refers to a chimeric molecule formed from a translocating peptide and a therapeutic or diagnostic agent. The peptide and agent are covalently linked, and this distinguishes the conjugates of the present invention from those in the prior art, which rely on non-covalent binding to DNA. The covalent linkage may be in the form of a chemical linker molecule, or may be in the form of a fusion protein.

The term “peptide” used herein, is intended to refer to both peptides and proteins.

The present invention is based on the surprising finding that histones are capable of undergoing translocation across a cell membrane. In particular, a critical amino acid sequence has now been identified as critical for successful translocation. The critical sequence is:

Lys Lys X¹ X² Lys SEQ ID NO. 1

where X¹ is preferably Alanine or Proline; and

X² is Lys or Arginine.

Identifying this sequence enables many different peptides to be produced, not just those derived from histones.

However, histones are preferred, particularly human histones. Although all the histones comprising SEQ ID NO. 1 may be used, it is preferred that human histone H1 is used. H1 histones exist in many different isoforms, although high levels of sequence homology exist between these. The amino acid sequence of a suitable human H1 histone is identified in Albig et al., Genomics, 1991; 10(4): 940-948. The sequences are also available on the NCBI database (GeneBank Accession No. M60748).

The histone may also be in a truncated form, preferably in a form identified below. Having the histone in a truncated form allows synthetic forms to be produced readily, without the need to undergo time-consuming and expensive purification steps. It was also found that truncated forms are produced more readily in recombinant expression systems, i.e. in a recombinant mammalian or bacterial expression system. In addition, truncated forms of the histones (or any small peptide) may be less immunogenic and therefore more suitable for administration of the therapeutic agent.

Functional variants of the histone proteins may also be used. For example, proteins with high levels (greater than 70%, preferably greater than 90%) of sequence similarity or identity are within the scope of the present invention. The variants may be produced using standard recombinant DNA techniques such as site-directed mutagenesis. The variants may also have conserved amino acid substitutions (although not in the critical amino acid sequence), e.g. replacement of a hydrophobic residue for a different hydrophobic residue. All this will be apparent to the skilled person, based on conventional protein technology. The variants must retain the functional ability to translocate across a cellular membrane.

In a preferred embodiment, the peptide fragment is no more than 50, preferably no more than 40, and most preferably no more than 30 amino acid residues. Peptides suitable for use in the invention comprise or consist of the sequences identified herein as SEQ ID NOS. 2 to 9.

The peptides may comprise the defined sequence motif more than once, for example two or three motifs may be present.

The peptides may also comprise a high percentage of Lys and Arg residues, typically greater than 5%, preferably more than 10%.

Sequences having conserved amino acid substitutions with histones, or the sequences identified herein (other than that of SEQ ID NO. 1), are also within the scope of the present invention. A skilled person will appreciate that conserved amino acid substitutions are those which, for example, replace one hydrophobic amino acid with a different hydrophobic amino acid.

In addition to the peptides identified herein, the conjugates comprise a discrete therapeutic or diagnostic agent. In the context of the present invention, a reference to “therapy” or “therapeutic agent” also includes prophylactic treatments, e.g. vaccination. Examples of suitable therapeutic and diagnostic agents include polynucleotides, proteins, peptides, antibodies, enzymes, antigens growth factors, hormones and contrast agents.

A protein therapeutic agent is preferably at least 100 amino acids in length. The present invention is particularly useful for longer sequences, e.g. at least 150, 200, 300, 400 or 1000 amino acids in length. For the avoidance of doubt, the term “protein” as used herein also encompasses polypeptides of the required length; although the term “polypeptide” generally means sequences of from 2 to 100 amino acids in length, usually 2 up to 60.

The therapeutic agent may comprise nucleic acid, e.g. a reporter gene. The nucleic acid may be DNA or RNA.

The nucleic acid may encode a therapeutic agent, e.g. an enzyme, toxin, immunogen, etc. or may itself be the therapeutic agent. For example, anti-sense RNA may be used to target and disrupt expression of a gene. All this will be apparent to the skilled person.

The therapeutic agent may also be a chemical compound, i.e. an organic or inorganic molecule. Any suitable pharmacological agent is within the scope of the present invention. Preferred chemical molecules include cytoxic agents and growth factors.

It is preferred if the therapeutic agent is not a neuropeptide that has its site of action outside of the cell. A neuropharmacologic agent may be used as part of the conjugate, but it should exert its therapeutic effect intracellularly. Therefore, neuropeptides which act extracellularly, e.g. on cell surface receptors, are not preferred. It is apparent that the conjugates of the invention are intended for translocation, and therefore the site of action of the therapeutic agent will be within the cell.

The conjugates of the invention may be produced via techniques known to those skilled in the art. The peptide and agent are linked via a covalent attachment. In one embodiment the agent is a peptide (or protein) and the conjugate is a fusion protein. The production of fusion proteins is known to those skilled in the art and comprises the production of a recombinant polynucleotide that encodes, in frame, both the peptide and the agent. For example, nucleic acid encoding a suitable conjugate may be incorporated into a suitable expression vector for further manipulation. As used herein, vector (or plasmid) refers to discrete elements that are used to introduce heterologous DNA into cells for either expression or replication thereof. Selection and use of such vehicles are well known to the skilled person. Many vectors are available, and selection of appropriate vector will depend on the intended use of the vector, e.g. whether it is to be used for DNA amplification or for DNA expression, the size of the DNA to be inserted into the vector, and the host cell to be transformed with the vector. Each vector contains various components depending on its function (amplification of DNA or expression of DNA) and the host cell for which it is compatible. The vector components generally include, but are not limited to, one or more of the following: an origin of replication, one or more marker genes, an enhancer element, a promoter, a transcription termination sequence and a signal sequence.

The conjugates may also be produced by the use of bifunctional reagents which are capable of reacting with the peptide and agent. For example, conjugation of the peptide and agent may be achieved by reagents such as N-succinimidyl 3-(2-pyridyl-dithio)propionate (SPDP) which form a disulphide bridge. Alternative conjugation reagents include: glutaraldehyde, cystamine and EDAC.

Preferably, the agent is linked by a cleavable linker region to the peptide region. Preferably, the cleavable linker region is a protease-cleavable linker, although other linkers, cleavable for example by small molecules, may be used. These include Met-X sites, cleavable by cyanogen bromide, Asn-Gly, cleavable by hydroxylamine, Asp-Pro, cleavable by weak acid, and Trp-X, cleavable by, inter alia, NBS-skatole. Protease cleavage sites are preferred due to the milder cleavage conditions necessary and are found in, for example, factor Xa, thrombin and collagenase. Any of these may be used. The precise sequences are available in the art and the skilled person will have no difficulty in selecting a suitable cleavage site. By way of example, the protease cleavage region targeted by Factor Xa is I E G R. The protease cleavage region targeted by Enterokinase is D D D D K. The protease cleavage region targeted by Thrombin is L V P R G. Preferably, the cleavable linker region is one which is targeted by endocellular proteases.

Additional cell transportation signals may be present. For example, nuclear localisation signals may be an additional component of the constructs. This may aid the transport of the therapeutic component to the correct intracellular location. Suitable signals are-known and identified in the prior art.

It is apparent that the compositions and constructs of the invention are intended for therapeutic use.

Applications for the conjugates of the present invention include:

1. Antigen delivery system.

1. An antigen is any agent that when introduced into an immunocompetent animal stimulates the production of a specific antibody or antibodies that can combine with the antigen. However, the antigen may need to be combined with a carrier to be able to stimulate antibody production or specific T cells (helper or cytotoxic). This is where the present invention may be useful as a carrier for transporting the antigen from one side of the cell membrane to the other such that it can stimulate antibody production. By way of example, bacterial and viral antigens translocated by the conjugates in the cell cytoplasm may be processed and associated with MHC class 1 molecules. This antigen processing and presenting pathway is known to activate specific CD8 cytoxic lymphocytes.

2. Gene therapy.

Gene therapy may include any one or more of: the addition, the replacement, the deletion, the supplementation, the manipulation etc. of one or more nucleotide sequences in, for example, one or more targeted sites—such as targeted cells. If the targeted sites are targeted cells, then the cells may be part of a tissue or an organ. General teachings on gene therapy may be found in Molecular Biology, Ed Robert Meyers, Pub VCH, such as pages 556-558.

By way of further example, gene therapy can also provide a means by which any one or more of: a nucleotide sequence, such as a gene, can be applied to replace or supplement a defective gene; a pathogenic nucleotide sequence, such as a gene, or expression product thereof can be eliminated; a nucleotide sequence, such as a gene, or expression product thereof, can be added or introduced in order, for example, to create a more favourable phenotype; a nucleotide sequence, such as a gene, or expression product thereof can be added or introduced, for example, for selection purposes (i.e. to select transformed cells and the like over non-transformed cells); cells can be manipulated at the molecular level to treat, cure or prevent disease conditions such as cancer (Schmidt-Wolf and Schmidt-Wolf, 1994, Annals of Hematology 69; 273-279) or other disease conditions, such as immune, cardiovascular, neurological, inflammatory or infectious disorders; antigens can be manipulated and/or introduced to elicit an immune response, such as genetic vaccination. In a particularly preferred embodiment, the compositions may be used to introduce functional proteins in the cytoplasm of genetically deficient cell types.

3. Cancer therapy.

The compositions may be used to transport into cancer cells molecules that are transcription factors and are able to restore cell cycle control or induce differentiation. For example, it is understood that many cancer cells would undergo apoptosis if a functional P-53 molecule is introduced into their cytoplasm. The present invention may be used to deliver such gene products.

4. Antibacterial and antiviral therapy.

For example, the compositions may be used to transport in the cytoplasm of infected cells recombinant antibodies or additional DNA-binding molecules which interfere with a crucial step of bacterial and viral replication.

5. Use in expression systems.

For example, it is desirable to express exogenous proteins in eukaryotic cells so that they get processed correctly. However, many exogenous proteins are toxic to eukaryotic cells. In manufacturing exogenous proteins it is therefore desirable to achieve temporal expression of the exogenous protein. The system may therefore be used in connection with an inducible promoter for this or any other application involving such a system.

6. Protein sorting.

7. DNA synthesis.

8. Contrast imaging

A suitable contrast agent may be part of the conjugate to allow imaging to be carried out.

A composition comprising the conjugates of the invention may optionally comprise a pharmaceutically acceptable carrier, diluent, excipient or adjuvant. The choice of pharmaceutical carrier, excipient or diluent can be selected with regard to the intended route of administration and standard pharmaceutical practice. The pharmaceutical compositions may further comprise any suitable binder(s), lubricant(s), suspending agent(s), coating agent(s), solubilising agent(s), and other carrier agents that may aid or increase entry into the target site.

The compositions may be adapted for any route of administration, including intramuscular, intravenous, intradermal or subcutaneous. It is preferred if the compositions are free of serum, calcium and chloroquine. This is a further distinction from the prior art where serum, calcium or chloroquine is present during the transfection process.

The delivery of one or more therapeutic genes or proteins according to the invention may be carried out alone or in combination with other treatments or components of the treatment. Diseases which may be treated include, but are not limited to: cancer, neurological diseases where the agent is required intracellularly, inherited diseases, heart disease, stroke, arthritis, viral infections and diseases of the immune system. Suitable therapeutic genes include those coding for tumour-suppressor proteins, enzymes, pro-drug activating enzymes, immunomodulatory molecules, antibodies, engineered immunoglobulin-like molecules, conjugates, hormones, membrane proteins, vasoactive proteins or peptides, cytokines, chemokines, anti-viral proteins, antisense RNA and ribozymes.

The amount to be administered to a patient will depend on the usual factors: age of the patient, weight, severity of the condition, route of administration, activity of the therapeutic etc. All this can be determined by conventional methods known to the skilled person.

The following Examples illustrate the invention.

### EXAMPLE 1

The histone proteins used in this Example were all derived from human linker Histone H1 (GeneBank Accession No. M60748). The H1.4 fragment and sub-fragments (identified as SEQ ID NOS. 2 to 9) were cloned into a bacterial expression vector. For detection and purification, a tag was inserted at the N- and C-terminal part of the histone gene or its sub-fragments. A 6× histidine tag was placed at the N-terminus of the histone sequence and was used to purify the desired protein by affinity chromatography through a Nickel column (Quiagen). The second tag corresponds to the c-myc epitope (a known peptide tag obtainable from commercial sources) and was used for immuno-fluorescence detection of the recombinant protein.

Each recombinant protein was purified under denaturing conditions through a Nickel column and dialysed against 20 mM Tris-HCl pH8, 0.5 m NaCl and 0.1% tween 20 except for the full length histone H1.4 which required a dialysis buffer of 0.1 M phosphate buffer pH4.7.

Translocation procedure:

HeLa cells were seeded at 50-80% confluence in RPMI 1640 either supplemented by 10% FCS or in the absence of FCS. Each protein was added to the cell media and left to incubate for 3 hours at 37° C. Translocation of the protein was determined by immuno-detection using the c-Myc antibody (Sigma).

It was found that those peptides comprising SEQ ID NO. 1 were able to translocate across the plasma membrane.

### EXAMPLE 2

In a separate experiment, two synthetic peptides were produced, and covalently attached to a biotin molecule. The sequences were:

SEQ ID NO. 10

Biotin-spacer-TPKKASSPAAAAGAKKAKSP-amide

SEQ ID NO. 11

Biotin-spacer-TPKKAKKPAAAAGASSAKSP-amide

The synthetic peptides were assessed for translocation by reacting the biotin tag with fluorescently-labelled avidin.

SEQ ID NO. 10 was used to test whether substituting SS for KK in the sequence motif altered the translocation efficiency. Using this sequence, no translocation was observed.

SEQ ID NO. 11 contained the sequence motif, and also two further S residues that substituted further K residues. Translocation was observed.

1. A conjugate of a peptide capable of translocation across a cell membrane, and a therapeutic or diagnostic agent, wherein the peptide comprises the amino acid sequence KKX¹X²K, where X¹=A or P and X²═K or R.

2. A conjugate according to claim 1, wherein the agent is a therapeutic protein.

3. A conjugate according to claim 1, wherein the agent is not a neuropeptide.

4. A conjugate according to claim 1, wherein the agent is a polynucleotide molecule.

5. A conjugate according to claim 1, wherein the agent is a contrast agent.

6. A conjugate according to claim 1 or claim 2, wherein the agent has its site of therapeutic activity within a cell.

7. A conjugate according to any of claim 1 to 3, which is a fusion protein.

8. A conjugate according to any of claims 1 to 4, wherein the agent is conjugated to the peptide via a chemical linker molecule.

9. A conjugate according to any preceding claim, wherein the peptide comprises at least 20 amino acids.

10. A conjugate according to any preceding claim, wherein the peptide is a histone, or a fragment thereof.

11. A conjugate according to any preceding claim, wherein the peptide is human histone H1, or a fragment thereof.

12. A conjugate according to any preceding claim, wherein the peptide comprises or consists of any of the histone fragments identified herein as SEQ ID NOS. 2 to 9.

13. A conjugate according to any preceding claim, wherein the peptide is a recombinant peptide.

14. A conjugate according to any preceding claim, for use in therapy or diagnosis.

15. Use of a therapeutic agent that exerts its therapeutic effect within a cell, in the manufacture of a composition to treat a disease, wherein the agent is conjugated to a peptide that comprises the amino acid sequence defined in claim 1.

16. Use of a conjugate of a peptide capable of translocation across a cell membrane and a therapeutic or diagnostic agent, in the manufacture of a composition to treat or diagnose a disease, wherein the peptide comprises the amino acid sequence defined in claim 1, with the proviso that the disease is not a neurological disorder.

17. Use according to claim 15 or claim 16, wherein the conjugate is as defined in any of claims 2 to 13.

18. Use according to any of claims 15 to 17, for intramuscular administration.

19. Use according to any of claims 15 to 18, wherein the medicament does not comprise calcium, serum or chloroquine.

20. An expression vector that encodes a fusion protein according to claim 7.

21. A recombinant cell line comprising an expression vector according to claim 20.

22. A process for the production of a therapeutic or diagnostic agent capable of being delivered across a cell membrane, comprising covalently attaching the agent to a peptide as defined in claim 1.
